(19) United States
(12) Patent Application Publication
(10) Pub. No.: US 2005/0177029 A1
(43) Pub. Date: Aug. 11, 2005

(54) Earphone-type physiological function detecting system

(76) Inventor: Yuan-Yao Shen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/774,510

(22) Filed: Feb. 10, 2004

(51) Int. Cl. A61B 5/00; A61B 6/00; A61B 5/05; B65D 81/00

(52) U.S. Cl. 600/300; 128/903; 128/904

(57) ABSTRACT

An earphone-type physiological function detecting system is provided in the present invention. In which, a detecting sensor module is disposed on a detecting unit, so that the detecting sensor module of the detecting unit is able to detect various the physiological function, such as the body temperature, the blood pressure, the heartbeat, the blood sugar of the user when the user wears the detecting unit in the ear. Further, the detecting unit transmits the detected physiological function signals to the devices, such as the mobile phone, the MP3 walkman, the CD walkman, and the radio for displaying. When the detected signals exceed the standard values, the physiological function detecting system rings as an alarm, stores the detected signals in a memory module, transmits the detected signals to the far end for displaying, and records the daily detected values.

### BACKGROUND OF THE INVENTION

1. Field of the Invention

This invention relates to an earphone-type physiological function detecting system, and particularly to an earphone-type physiological function detecting system, which is able to detect various physiological function of the users, transmit and display the detected signals to a portable device having a display panel thereon.

2. Description of the Prior Art

Severe Acute Respiratory Syndrome (SARS) is a legal infectious disease raised and developed from February 2003. The main infectious routes of SARS are via the respiratory droplets and the body fluids produced by the infected persons. Once people closely contact with the carrier of SARS, it is very easy to be infected with SARS. Further, it is possible that the infected persons may be dead when the situation is serious. The main symptoms of SARS include high fever (>38° C.), dry cough, tachypnoea, dyspnea, lung pathological changes and go along with other symptoms. In which, the high fever is the first symptom of being infected with SARS.

In order to avoid the infection sources of SARS virus from spreading continuously, first of all is to centralize and treat all the infected persons. However, the only way to find out the infected persons is to sieve them from the persons having fevers. Therefore, to measure the body temperature is a very important task for the moment. If everyone can measure his body temperature regularly every day, it is possible to timely know whether he has a fever or not, and to find out the infected persons effectively. But, for achieving the above, the user needs to carry a temperature meter anytime, which is inconvenient to the user. For this reason, if the ear thermal meter can be combined with the portable devices, such as the mobile phone, the MP3 walkman, the CD walkman, the radio and so forth, the above defects will be overcome.

This conception can be further expanded to a physiological function detecting device for detecting the blood pressure, the heartbeat, the blood sugar and etc., and then the goal of the body health control is achieved by the functions of displaying, alarming and transmitting of the mobile phone.

### SUMMARY OF THE INVENTION

It is an object of the present invention to provide an earphone-type physiological function detecting system. In which, a detecting unit having a detecting sensor module therein is provided to detect various physiological function of the users by the detecting unit, and the physiological function detecting system is combined to the earphones of the devices such as the mobile phone, the MP3 walkman, the CD walkman, and the radio for displaying the detected physiological function values.

It is another object of the present invention to design a device, which can detect the physiological function signals, into an earphone type or to design the device into an earphone-type physiological function detecting system combined with the earphones of the devices such as the mobile phone, the MP3 walkman, the CD walkman, the radio.

It is a further object of the present invention to provide an earphone-type physiological function detecting system, which will alarm by ringing while the users have unusually high physiological function and fevers.

It is an additional object of the present invention to provide a design of attaching a detecting unit into the earphones of the devices such as the mobile phone, the MP3 walkman, the CD walkman, and the radio, so that the earphones of the devices can not only receive but also detect various physiological function of the users. Therefore, when the users go out, they can detect their physiological function anytime and have no obstacle for carrying.

The earphone-type physiological function detecting systems, which can achieve the above objects, are the devices have a detecting unit with a detecting sensor module therein. The detecting unit is able to be worn in the user's ear to detect the physiological function such as the body temperature, the blood pressure, the heartbeat, the blood sugar and so forth. Further, the detecting unit can convert the detected physiological function signals into the signals receivable to the mobile phone, the MP3 walkman, the CD walkman and the radio by a signal converting module, and then it can send the signals to the mobile phone for displaying. Once the detected signals exceed the standard values, the buzzers of the mobile phone, the MP3 walkman, the CD walkman or the radio will ring as an alarm, and the users can know their physiological function anytime. Further, the detected physiological function values can be transmitted to the far ends via the transmission interfaces or the detected signals can be stored in the memory modules for recording the detected values of each day.

### BRIEF DESCRIPTION OF THE DRAWINGS

The drawings disclose an illustrative embodiment of the present invention, which serves to exemplify the various advantages and objects hereof, and are as follows:

FIG. 1 shows the structural blocks of the earphone-type physiological function detecting system of the present invention;

FIG. 2 shows the diagram of the earphone-type physiological function detecting system according to a first embodiment of the present invention;

FIG. 3 shows the diagram of the earphone-type physiological function detecting system according to a second embodiment of the present invention;

FIG. 4 shows the another block diagram of the earphone-type physiological function detecting systems of the present invention; and

FIGS. 5A and 5B show the diagrams of the earphone-type physiological function detecting system according to a third embodiment of the present invention.

### DETAILED DESCRIPTION OF THE PREFERRED EMBODIMENT

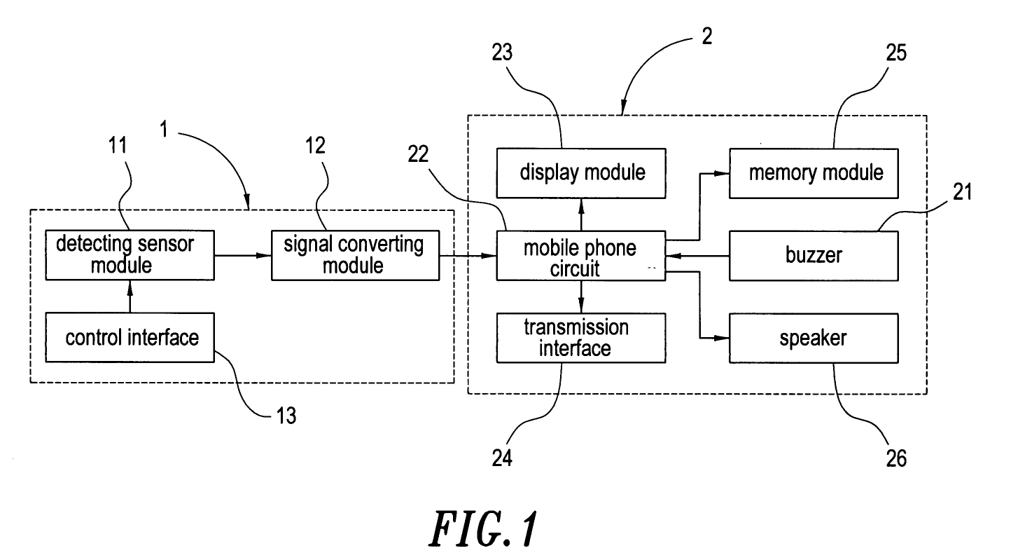

Please refer to FIG. 1 showing the block diagram of the earphone-type physiological function detecting system according to a first embodiment of the present invention. The earphone-type physiological function detecting system mainly includes a detecting unit 1, which has the detecting sensor module 11, the signal converting module 12, and the control interface 13 combined therein. In which, the control interface 13 is able to control the switch of the detecting sensor module 11. The detecting sensor module 11 is able to be set on either the detecting unit 1 or any contact surface of the human body, and it is used to detect the physiological function signals such as the body temperature, the heartbeat, the blood sugar, the blood pressure, and so forth of the detected human body. Further, the detecting unit 1 transmits the detected signals to the signal converting module 12. After converted by the signal converting module 12, the signals become the converted signals receivable to the mobile phone 2, and are transmitted to the mobile phone 2. The mobile phone 2 includes a control interface 21, a mobile phone circuit 22, a display module 23, a transmission interface 24, a memory module 25, and a buzzer 26. In which, the control interface 21 is connected to the mobile phone circuit 22 for transmitting a control signal to the mobile phone circuit 22 and controls all the input/output module actions. Further, the control interface 21 can be the key sets on the mobile phone 2. The mobile phone circuit 22 receives the signals from the signal converting module 12 of the detecting unit 1 and the signals from the control interface 21, identifies the signals, and transmits the signals to each output modules to be executed. The display module 23 receives the physiological function signals from the mobile phone circuit 22 and displays the received signals. The transmission interface 24 receives the physiological function signals from the mobile phone circuit 22, and transmits the received signals to the mobile phone 2 located on the far end via a base station for displaying. The memory module 25 receives the physiological function signals from the mobile phone circuit 22 and stores the received signals for recording the signals completely. The buzzer 26 receives the physiological function signals from the mobile phone circuit 22. Once the received signals exceed the standard values, the buzzer 26 rings as an alarm for reminding the user that the received signals are overhigh, in which, the alarm can be the built-in ring of the mobile phone 2.

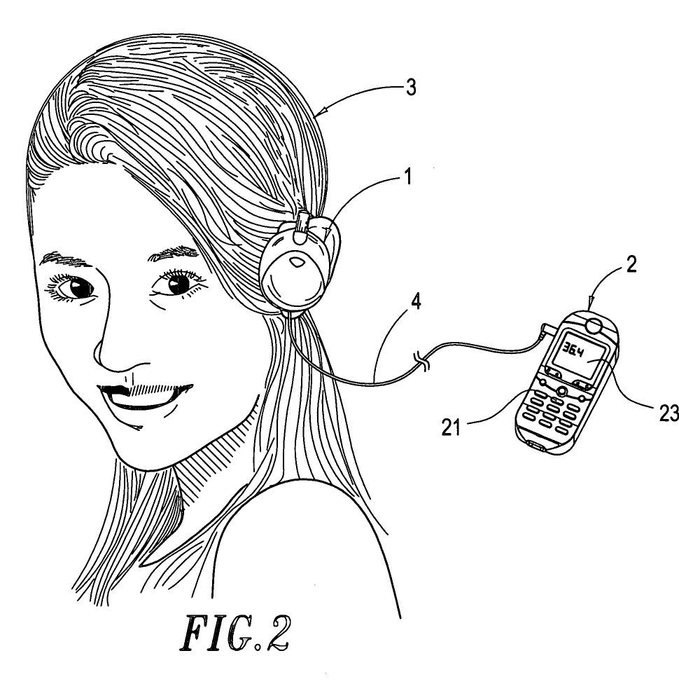

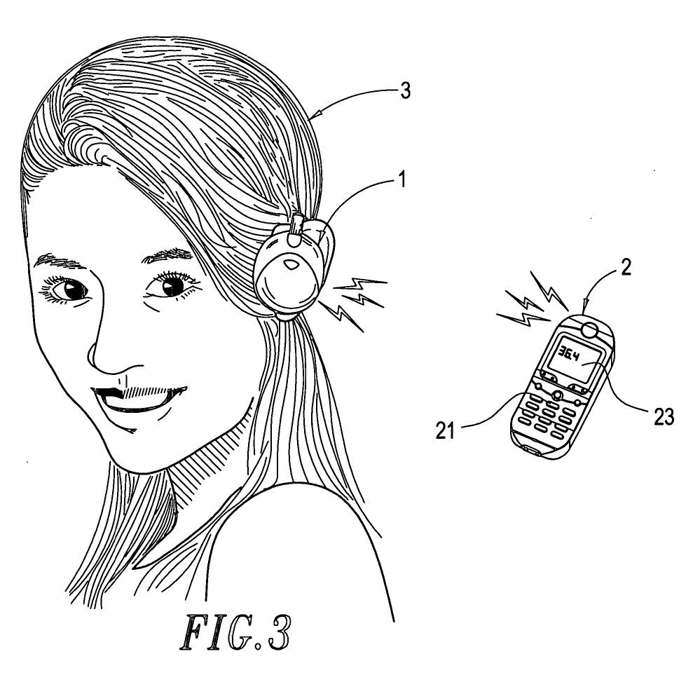

Further, please refer to FIGS. 2 and 3 illustrating the embodiments of FIG. 1. When the user 3 wears the detecting unit on the ear, it is practicable to control the switch of the detecting sensor module 11 by the control interfaces 13, 21 of the detecting unit 1 and the mobile phone 2. If the switch of the detecting sensor module 11 is on, the detecting sensor module 11 of the detecting unit 1 detects the physiological function signals of the user 3 such as the body temperature, the blood pressure, the blood sugar, the heartbeat, and so forth. Then, the detecting sensor module 11 transmits the detected signals to the signal converting module 12 for converting them into the signals receivable to the mobile phone 2. Further, the converted body temperature signals are transmitted to the mobile phone circuit 22 of the mobile phone 2 via a transmission line 4. After receiving the converted signals, the mobile phone circuit 22 transmits the converted physiological function signals to the display module 23 for displaying. When the physiological function signals exceed the standard values, the mobile phone circuit 22 drives the buzzer 26 to ring as an alarm in order to remind the user that the physiological function are overhigh and immediately taking a suitable medical treatment is necessary. In addition, the user stores the detected physiological function values in the memory module 25 via the control interface 21 or transmits the detected physiological function values to the far end via the transmission interface 24 for showing them to other persons. Additionally, as shown in FIG. 3, the detected physiological function signals can be transmitted from the detecting unit 1 to the mobile phone 2 via the wireless transmissions such as the bluetooth, the infrared, the radio frequency and so forth.

Further, since the detecting unit 1 is able to be built in the special earphones of the devices such as the mobile phone, the MP3 walkman, the CD walkman, and the radio, the earphones can not only be used to receive but also to detect the physiological function of the user 3, such as the body temperature, the blood pressure, the blood sugar, the heartbeat, and so forth.

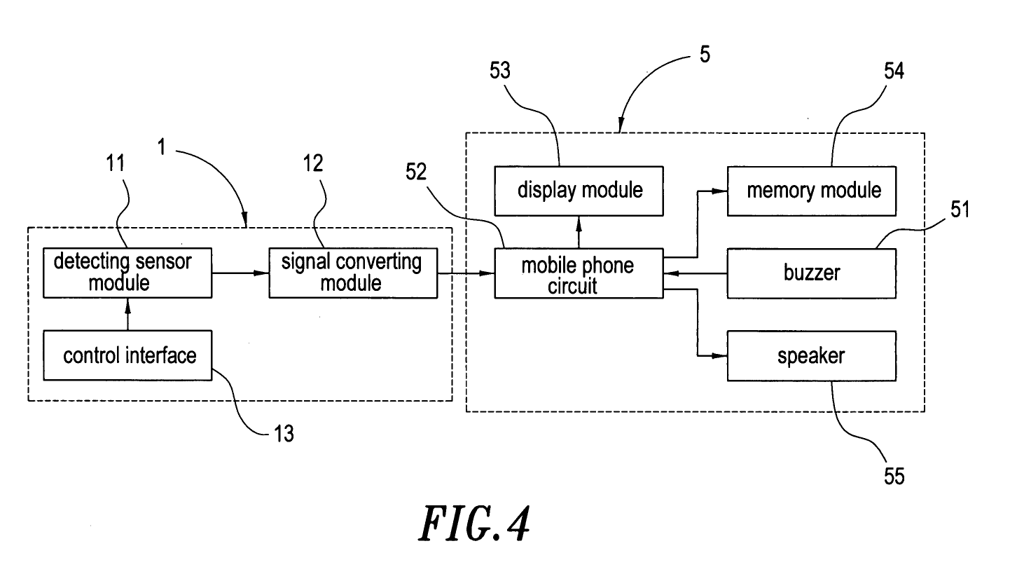

Please refer to FIG. 4 illustrating the block diagrams of the earphone-type physiological function detecting system according to the second embodiment of the present invention. The earphone-type physiological function detecting system mainly includes a detecting unit 1, which has the detecting sensor module 11, the signal converting module 12, and the control interface 13 combined therein. In which, the control interface 13 is able to control the switch of the detecting sensor module 11. The detecting sensor module 11 is able to be set on either the detecting unit 1 or any contact surface of the human body, and it is used to detect the physiological function signals of the detected human body, such as the body temperature, the heartbeat, the blood sugar, the blood pressure, and so forth. Further, the detecting unit 1 transmits the detected signals to the signal converting module 12. After converted by the signal converting module 12, the detected signals become the converted signals receivable to the device, such as the CD walkman 5, the MP3 walkman or the radio. Then the converted signals are transmitted to the device, such as the CD walkman 5, the MP3 walkman or the radio. The device, such as the CD walkman 5, the MP3 walkman or the radio, includes a control interface 51, a receiving circuit 52, a display module 53, a memory module 54, and a speaker 55. In which, the control interface 51 is connected to the receiving circuit 52 for transmitting a control signal to the receiving circuit 52 and controls all the input/output module actions. Further, the control interface 51 can be the key sets on the device, such as the CD walkman 5, the MP3 walkman or the radio. The receiving circuit 52 receives the signals from the signal converting module 12 of the detecting unit 1 and the signals from the control interface 51, identifies the received signals, and transmits the identified signals to each output modules to be executed. The display module 53 receives the physiological function signals from the receiving circuit 52 and displays the received signals. The memory module 54 receives the physiological function signals from the receiving circuit 52 and stores the received signals for completely recording the signals. The speaker 55 receives the physiological function signals from the receiving circuit 52. Once the received signals exceed the standard values, the speaker 55 rings as an alarm for reminding the user that the received signals are overhigh. Namely, the user can know and control his own physiological function anytime, if necessary.

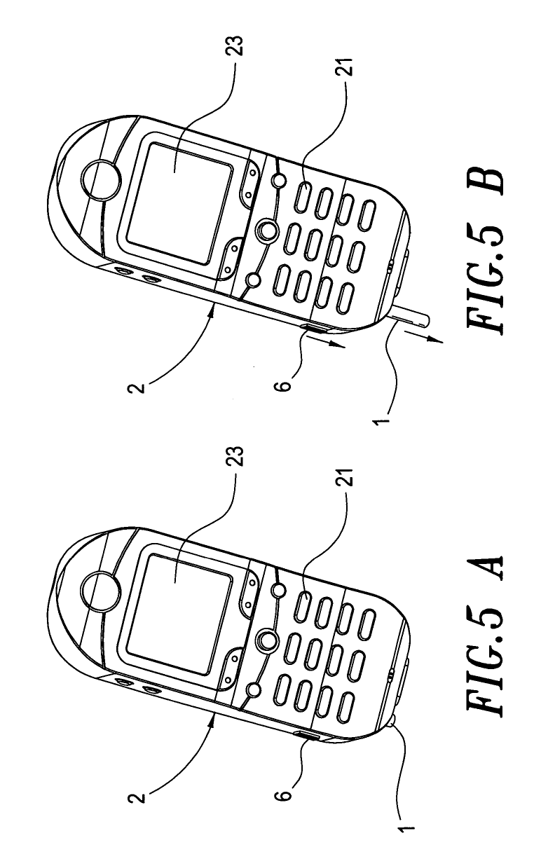

In addition, please refer to FIGS. 5A and 5B. The detecting unit 1 is able to be combined with the devices such as the mobile phone 2, the CD walkman 5, the MP3 walkman, and the radio. To give an example of the mobile phone 2, the detecting unit 1 is hidden in the mobile phone 2, and a push button 6 is disposed on the side edge of the mobile phone 2. The push button 6 is connected with the detecting unit 1, and is able to control the detecting unit 1 being inserted or not. Since the detecting unit 1 is directly attached on the mobile phone 2, it is possible for the user to carry the device therewith, such as the mobile phone 2, the CD walkman 5, the MP3 walkman and the radio, so as to detect his own physiological function anytime.

Further, the detecting unit 1 can also be set apart from the devices such as the mobile phone 2, the CD walkman 5, the MP3 walkman, and the radio. When it is to be used, the detecting unit 1 is inserted into the slot of the device, such as the mobile phone 2, the CD walkman 5, the MP3 walkman, or the radio, for detecting the physiological function of the user.

Compared with the prior art, the earphone-type physiological function detecting system according to the present invention further has the following advantages:
1. The present invention disposes a detecting sensor module into a detecting unit. Therefore, when the user wears the detecting unit on his ear, the detecting unit is able to be used to detect the various physiological function of the user and then transmit the detected signals to the devices, such as the mobile phone, the MP3 walkman, the CD walkman and the radio, for displaying. Accordingly, the user can know and control his physiological function anytime.
2. The physiological function signals detected by the earphones of the present invention can be transmitted to the far end via the mobile phone, or stored in the mobile phone for the user to make a record.
3. The physiological function detecting system of the present invention alarms the user that the detected physiological function values are overhigh by ringing, and if the user has a fever, a medical treatment can be taken immediately.
4. It is possible to dispose a detecting unit into the special earphones of the devices, such as the mobile phone, the MP3 walkman, the CD walkman, or the radio, according to the present invention, and then the earphones can be used to detect the physiological function of the human body, except the receiving functions.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. An earphone-type physiological function detecting system, which attaches a physiological function detecting unit into a portable electronic product for a user to detect his physiological functions anytime and anywhere, and displays, alarms, stores, and transmits a detected result to a far end by said portable electronic product.

2. The earphone-type physiological function detecting system according to claim 1, wherein said portable electronic product is one selected from a group consisting of a mobile phone, a MP3 walkman, a CD walkman, and a radio.

3. An earphone-type physiological function detecting system, comprising:
a detecting unit with a detecting sensor module and a signal converting module combined therein, in which said signal converting module receives a physiological function signal from said detecting sensor module, converts said signal into a mobile phone receivable signal, and transmits said signal to said mobile phone via a wired way or a wireless way;
said mobile phone comprising:
a control interface connecting with a mobile phone circuit for transmitting a control signal to said mobile phone circuit and controlling each input/output module action accordingly;
a mobile phone circuit receiving signals from said signal converting module and said control interface, identifying said signals, and transmitting said signals to each output module to be executed;
a display module receiving physiological function signals from said mobile phone circuit and displaying said signals;
a transmission interface receiving physiological function signals from said mobile phone circuit for transmitting said signals to a far end;
a memory module receiving physiological function signals from said mobile phone circuit for storing said signals; and
a buzzer receiving signals from said mobile phone circuit and ringing as an alarm for reminding that detected physiological function signals exceed standard values.

4. The earphone-type physiological function detecting system according to claim 3, wherein said detecting unit also directly combines with said mobile phone, and a push button is disposed on a panel of said mobile phone, in which said push button controls said detecting unit being inserted or not.

5. The earphone-type physiological function detecting system according to claim 3, wherein said detecting unit is disposed apart from said mobile phone, and said detecting unit is inserted into a slot of said mobile phone for detecting said physiological function signal, when being used.

6. An earphone-type physiological function detecting system, comprising:
a detecting unit with a detecting sensor module and a signal converting module combined therein, in which said signal converting module receives a physiological function signal from said detecting sensor module, converts said signal into an MP3 walkman receivable signal and transmits said signal to said MP3 walkman via a wired way or a wireless way;
said MP3 walkman comprising:
a control interface connecting with a receiving circuit for transmitting a control signal to said receiving circuit and controlling each input/output module action accordingly;
a receiving circuit receiving signals from said signal converting module and said control interface, identifying said signals, and transmitting said signals to each output module to be executed;
a display module receiving physiological function signals from said receiving circuit and displaying said signals;
a memory module receiving physiological function signals from said receiving circuit for storing said signals; and
a speaker receiving signals from said receiving circuit and ringing as an alarm for reminding that detected physiological function signals exceed standard values.

7. The earphone-type physiological function detecting system according to claim 6, wherein said detecting unit also directly combines with said MP3 walkman, and a push button is disposed on a panel of said MP3 walkman, in which said push button controls said detecting unit being inserted or not.

8. The earphone-type physiological function detecting system according to claim 6, wherein said detecting unit is disposed apart from said MP3 walkman, and said detecting unit is inserted into a slot of said MP3 walkman for detecting said physiological function signal, when being used.

9. An earphone-type physiological function detecting system, comprising:
a detecting unit with a detecting sensor module and a signal converting module combined therein, in which said signal converting module receives a physiological function signal from said detecting sensor module, converts said signal into a CD walkman receivable signal, and transmits said signal to said CD walkman via a wired way or a wireless way;
said CD walkman comprising:
a control interface connecting with a receiving circuit for transmitting a control signal to said receiving circuit and controlling each input/output module action accordingly;
a receiving circuit receiving signals from said signal converting module and said control interface, identifying said signals, and transmitting said signals to each output module to be executed;
a display module receiving physiological function signals from said receiving circuit and displaying said signals;
a memory module receiving physiological function signals from said receiving circuit for storing said signals; and
a speaker receiving signals from said receiving circuit and ringing as an alarm for reminding that detected physiological function signals exceed standard values.

10. The earphone-type physiological function detecting system according to claim 10, wherein said detecting unit also directly combines with said CD walkman, and a push button is disposed on a panel of said CD walkman, in which said push button controls said detecting unit being inserted or not.

11. The earphone-type physiological function detecting system according to claim 9, wherein said detecting unit is disposed apart from said CD walkman, and said detecting unit is inserted into a slot of said CD walkman for detecting said physiological function signal, when being used.

12. An earphone-type physiological function detecting system, comprising:
a detecting unit with a detecting sensor module and a signal converting module combined therein, in which said signal converting module receives a physiological function signal from said detecting sensor module, converts said signal into a radio receivable signal, and transmits said signal to said radio via a wired way or a wireless way;
said radio comprising:
a control interface connecting with a receiving circuit for transmitting a control signal to said receiving circuit and controlling each input/output module action accordingly;
a receiving circuit receiving signals from said signal converting module and said control interface, identifying said signals, and transmitting said signals to each output module to be executed;
a display module receiving physiological function signals from said receiving circuit and displaying said signals;
a memory module receiving physiological function signals from said receiving circuit for storing said signals; and
a speaker receiving signals from said receiving circuit and ringing as an alarm for reminding that detected physiological function signals exceed standard values.

13. The earphone-type physiological function detecting system according to one of claim 12, wherein said detecting unit also directly combines with said radio, and a push button is disposed on a panel of said radio, in which said push button controls said detecting unit being inserted or not.

14. The earphone-type physiological function detecting system according to claim 12, wherein said detecting unit is disposed apart from said radio, and said detecting unit is inserted into a slot of said radio for detecting said physiological function signal, when being used.
